(19) United States
(12) Patent Application Publication
(10) Pub. No.: US 2026/0166173 A1
(43) Pub. Date: Jun. 18, 2026

(54) PEPTIDE TARGETING BRCA2 PROTEIN AND APPLICATION THEREOF, AND DRUG FOR TREATING PROSTATE CANCER

(71) Applicant: The First Affiliated Hospital of Xi'an Jiao Tong University, Xi'an (CN)

(72) Inventors: Lei Li, Xi'an (CN); Jian Ma, Xi'an (CN); Qi Ye, Xi'an (CN)

(21) Appl. No.: 19/357,357

(22) Filed: Oct. 14, 2025

(30) Foreign Application Priority Data
Dec. 16, 2024 (CN) 2024118517459

(51) Int. Cl. A61K 47/69; A61K 31/502; A61K 38/00; A61P 35/00; C07K 14/00

(52) U.S. Cl. CPC A61K 47/6929; A61K 31/502; A61P 35/00; C07K 14/001; A61K 38/00

(57) ABSTRACT

A peptide targeting a BRCA2 protein and an application thereof, and a drug for treating prostate cancer are provided, which relate to the technical field of protein peptides. The amino acid sequence of the peptide is shown in SEQ ID NO: 1. The peptide has strong binding ability with BRCA2 protein, and can deliver the BRCA2 protein into prostate cancer cells, which can effectively inhibit cell proliferation and tumor growth, and has an ability of targeted degradation of BRCA2. Sensitivity of prostate cancer cells to PARP inhibitor can be significantly enhanced by combining the peptide with PARP inhibitor. Thus, the peptide can be used in preparing the drug for treating mCRPC or in preparing a reagent for degrading BRCA2 protein. BRCA2-targeted PROTAC drug can provide a new treatment strategy for prostate cancer, and PARP targeting inhibitor is combined to provide PARP inhibitor synthesis lethality treatment effect for mCRPC patients.

### CROSS-REFERENCE TO RELATED APPLICATION

This application claims priority to Chinese Patent Application No. 202411851745.9, filed on Dec. 16, 2024, which is herein incorporated by reference in its entirety.

### TECHNICAL FIELD

The disclosure relates to the technical field of protein peptides, and more particularly to a peptide targeting a breast cancer gene 2 (BRCA2) protein and an application method thereof, and a drug for treating prostate cancer.

### STATEMENT REGARDING SEQUENCE LISTING

The sequence listing associated with this application is provided in text format in lieu of a paper copy and is hereby incorporated by reference into the specification. The name of the XML file containing the sequence listing is 25025MYZ-USP1-SL.xml. The XML file is 1,875 bytes; is created on Aug. 7, 2025; and is being submitted electronically via patent center.

### BACKGROUND

Metastatic castration-resistant prostate cancer (mCRPC) is an aggressive malignancy with a poor prognosis. Clinical trials have shown that poly(adenosine diphosphate-ribose) polymerase (PARP) inhibitors can significantly improve overall survival (OS), objective response rate (ORR), and progression-free survival (PFS) of patients with homologous recombination (HR) repair gene defects. Compared with mutations in other HR genes, the PARP inhibitors have shown significant efficacy in patients carrying BRCA2 mutations. Unfortunately, only 19.3% of patients carry HR gene mutations, of which only 13% are BRCA2 gene mutations.

Targeting peptides are short peptides or proteins with specific binding ability, which can selectively bind to specific targets. However, general targeting peptides can only bind to targets and have limited degradation effects on the targets.

### SUMMARY

In view of this, an objective of the disclosure is to provide a peptide targeting a BRCA2 protein and an application method thereof, and a drug for treating prostate cancer. The peptide has high binding ability with the BRCA2 protein, which can efficiently degrade the BRCA2 protein, and effectively inhibit proliferation of prostate cancer cells, and can be used to prepare a drug for treating the prostate cancer.

In the first aspect of the disclosure, a peptide targeting the BRCA2 protein is provided, and the amino acid sequence of the peptide is as shown in SEQ ID NO: 1.

The BRCA2 protein plays a vital role in maintaining genome integrity. As shown in FIG. 6, the BRCA2 protein participates in repair of double-strand deoxyribonucleic acid (DNA) breaks through a HR pathway. The BRCA2 protein interacts with a RAD51 protein to promote the RAD51 protein to load single-stranded DNA at DNA damage sites, thereby completing DNA strand exchange and high-fidelity DNA repair. BRCA2 gene mutations can lead to abnormal DNA double-strand repair, enhance sensitivity of the prostate cancer and other tumors to a PARP inhibitor, and achieve a pharmacological mechanism of synthetic lethality.

The peptide targeting the BRCA2 protein provided by the disclosure has the amino acid sequence as shown in SEQ ID NO: 1. The disclosure uses an isothermal titration calorimetry experiment to detect the binding ability of the peptide with the BRCA2 protein and a damage specific DNA binding (DDB1) protein. A dissociation constant (KD) of the peptide with the BRCA2 protein is 116 nanomoles per liter (nM), and a dissociation constant of the peptide with the DDB1 protein is in a range of 208 nM to 428 nM. On the one hand, the peptide achieves degradation of the BRCA2 protein by binding to the BRCA2 protein; and on the other hand, the peptide enhances the degradation of the BRCA2 protein in the prostate cancer by inducing the DDB1 protein to ubiquitinate the BRCA2 protein.

In the second aspect of the disclosure, a drug for treating cancer is provided, and the drug includes the peptide and a delivery system.

In an embodiment of the disclosure, the delivery system is one selected from the group consisting of nano-gold, nano-selenium, and liposomes.

In an embodiment, the drug further includes a PARP inhibitor.

In a further embodiment, the PARP inhibitor includes Olaparib.

In the third aspect of the disclosure, an application method of the peptide in preparing a reagent for degrading the BRCA2 protein is provided.

In the fourth aspect of the disclosure, an application method of the peptide or the drug in preparing a drug for treating solid tumors is provided.

In an embodiment, the solid tumors include the prostate cancer.

In a further embodiment, the prostate cancer is mCRPC.

The disclosure has the following beneficial effects.

The disclosure delivers the peptide targeting the BRCA2 protein into the prostate cancer cells, which can effectively inhibit cell proliferation. It can be seen that the peptide can be used in preparing the drug for treating the prostate cancer or in preparing the reagent for degrading the BRCA2 protein.

The disclosure further provides a drug for treating cancer, which includes the peptide and the delivery system. The drug can provide a new treatment strategy for drug-resistant and advanced prostate cancer tumors, especially can solve a problem that the PARP inhibitor is not effective in patients who do not carry HR gene mutations, and fill the gap in targeted degradation drugs for the peptide targeting the BRCA2 protein worldwide.

### BRIEF DESCRIPTION OF DRAWINGS

FIGS. 1A-1E illustrate schematic diagrams of design results of a peptide targeting a BRCA2 protein according to the disclosure; in which:

FIG. 1A is a complex structure of the peptide targeting the BRCA2 protein and the BRCA2 protein;

FIG. 1B is a design result of a predicted sequence mutation of the BPD peptide;

FIG. 1C is an affinity detection result of the BPD peptide and the BRCA2 protein;

FIG. 1D is an affinity detection result of the BPD peptide and a DDB1 protein; and

FIG. 1E is a protein stability detection result of BPD relative to common peptides.

FIGS. 2A-2G illustrate schematic diagrams of detection results of in vitro degradation of the BRCA2 protein by the BPD according to the disclosure; in which:

FIG. 2A is a result of using a western blot method to detect degradation of the BRCA2 protein in different cancer cells by the BPD;

FIG. 2B is a result of using the western blot method to detect the degradation of the BRCA2 protein in cancer cells by the BPD at different treatment times;

FIG. 2C is a quantitative plot of FIG. 2B;

FIG. 2D is a result of using the western blot method to detect the degradation of the BRCA2 protein in cancer cells by the BPD peptide combined with N-benzyloxycarbonyl-leucyl-leucyl-leucinal (MG132);

FIG. 2E is a result of using a cellular immunofluorescence imaging method to detect a degradation effect of the BRCA2 protein in C4-2 cells by the BPD;

FIG. 2F is a mean fluorescence intensity of the BRCA2 protein in the C4-2 cells; and

FIG. 2G is phosphorylated histone H2AX (γ-H2AX) fluorescence foci (a level of γ-H2AX fluorescent focus formation in cell indicates DNA damage of the cell) of each C4-2 cell.

FIGS. 3A-3N illustrate schematic diagrams of research results on a mechanism of action of the BPD according to the disclosure; in which:

FIG. 3A is a result of using the western blot method to detect binding of an exogenously overexpressed DDB1 protein and the BRCA2 protein in 293T cells in a presence of the BPD;

FIG. 3B is a result of using the western blot method to detect binding of an exogenously overexpressed BRCA2 protein and the DDB1 protein in the 293T cells in the presence of the BPD;

FIG. 3C is a result of using the western blot method to detect binding of an endogenously expressed DDB1 protein and the BRCA2 protein in the 293T cells in the presence of the BPD;

FIG. 3D is a result of using the western blot method to detect binding of an endogenously expressed BRCA2 protein and the DDB 1 protein in the 293T cells in the presence of the BPD;

FIG. 3E is a result of using the western blot method to detect ubiquitination degradation of the BRCA2 protein by the exogenously overexpressed DDB1 protein in combination with the BPD;

FIG. 3F is a result of using the western blot method to detect degradation of the BRCA2 protein in the C4-2 cells by short hairpin RNA targeting DDB1 protein (shDDB1) in combination with the BPD;

FIG. 3G is a result of using the western blot method to detect degradation of the BRCA2 protein in the C4-2 cells by shDDB1 in combination with the BPD for different times;

FIG. 3H is a quantitative plot of FIG. 3G;

FIG. 3I is a result of using the cellular immunofluorescence imaging method to detect degradation of the BRCA2 protein in the C4-2 cells by shDDB1 in combination with the BPD;

FIG. 3J is a mean fluorescence intensity of the DDB1 protein in the C4-2 cells treated by shDDB1 in combination with the BPD;

FIG. 3K is a mean fluorescence intensity of the BRCA2 protein in the C4-2 cells treated by shDDB1 in combination with the BPD;

FIG. 3L is a result of using the cellular immunofluorescence imaging method to detect degradation of the BRCA2 protein in the PC-3 cells by shDDB1 in combination with the BPD;

FIG. 3M is a mean fluorescence intensity of the DDB1 protein in the PC-3 cells treated by shDDB1 in combination with the BPD; and

FIG. 3N is a mean fluorescence intensity of the BRCA2 protein in the PC-3 cells treated by shDDB1 in combination with the BPD;

FIGS. 4A-4H illustrate schematic diagrams of inhibition effects of the BPD on DNA damage HR of prostate cancer cells detected by the cellular immunofluorescence imaging method; in which:

FIG. 4A is a result of using the cellular immunofluorescence imaging method to detect a situation that a control group and the BPD are recruited to nucleus to degrade the BRCA2 protein under an ionizing radiation (IR) condition;

FIG. 4B is a quantitative plot of FIG. 4A;

FIG. 4C is a result of using the cellular immunofluorescence imaging method to detect a level of formed fluorescence foci of a RAD51 protein in the C4-2 cells under the IR condition after treated by the control group and the BPD;

FIG. 4D is quantitative plot of FIG. 4C;

FIG. 4E is a result of using the cellular immunofluorescence imaging method to detect a level of formed fluorescence foci of a RAD51 protein in the PC-3 cells under the IR condition after treated by the control group and the BPD;

FIG. 4F is a quantitative plot of FIG. 4E;

FIG. 4G is a result of using the western blot method to detect protein expression levels of a BRCA1 protein and the BRCA2 protein in the PC-3 cells after treated by transfecting small interfering RNA (si) Control (negative control group), si BRCA1, si BRCA2 and the BPD; and

FIG. 4H is a result of using a DNA damage HR repair fluorescence detection system to detect efficiencies of HR repair function of the PC-3 cells after treated by transfecting si Control (negative control group), si BRCA1, si BRCA2 and the BPD; where treatments of the si BRCA1, si BRCA2 and the BPD significantly damage HR efficiencies of cells.

FIGS. 5A-5K illustrate schematic diagrams of therapeutic efficacy of the BPD combined with a PARP inhibitor detected by cell and animal experiments; in which:

FIG. 5A is a result of using the western blot method to detect degradation of the BRCA2 protein in the prostate cancer cells by the BPD combined with the PARP inhibitor;

FIG. 5B is inhibition of C4-2 cell growth by the control group and the BPD;

FIG. 5C is inhibition of PC-3 cell growth by the control group and the BPD;

FIG. 5D a result of inhibition of C4-2 cell growth by the PARP inhibitor and combination of the BPD and the PARP inhibitor;

FIG. 5E is inhibition of PC-3 cell growth by the PARP inhibitor and combination of the BPD and the PARP inhibitor;

FIG. 5F is growth inhibitory effects of the control group, the PARP inhibitor and the BPD on subcutaneous transplantation tumor of the prostate cancer cells;

FIG. 5G is a quantitative plot of FIG. 5F;

FIG. 5H is growth inhibitory effects of the control group, the PARP inhibitor and the BPD on Xenotransplanted tumors of patient sources;

FIG. 5I is a quantitative plot of FIG. 5H;

FIG. 5J is a result of γ-H2AX (an expression level of γ-H2AX can indicate a DNA damage level in tissues) immunostaining on tumors after drug treatment; and

FIG. 5K is an immunostaining score result for the tumors after drug treatment.

FIG. 6 illustrates a schematic diagram of an effect of BRCA2 targeting PROTAC.

### DETAILED DESCRIPTION OF EMBODIMENTS

The disclosure is described in detail below in conjunction with drawings and embodiments, but they should not be construed as limitations of the disclosure.

The disclosure provides a peptide targeting a BRCA2 protein, and the amino acid sequence of the peptide is as shown in SEQ ID NO: 1.

SEQ ID NO: 1:
LLDEEDDSEEGGSGGTSSLLFSLWE.

The disclosure has no special restrictions on a source of the peptide, and any peptide source known in the art can be used. In the embodiment of the disclosure, the peptide is synthesized by using a peptide solid phase synthesis method. The disclosure has no special restrictions on the peptide solid phase synthesis method, and any peptide synthesis method known in the art can be used, such as Fmoc-peptide synthesis. Fmoc protected amino acids were purchased from GL Biochemical, and O-benzotriazole-N,N,N′,N′-tetramethyl-uronium hexafluorophosphate (HBTU, C₁₁H₁₆N₅OPF₆) and 1-hydroxybenzotriazole (HOBT, C₆H₅N₃O) condensing agents were from Suzhou Highfine Biotechnology.

In the disclosure, an isothermal titration calorimetry experiment is used to detect a binding ability of the peptide with the BRCA2 protein. A dissociation constant (KD) of the peptide with the BRCA2 protein is 116 nM. The peptide binds to the BRCA2 protein and a DDB1 protein to induce the DDB1 protein to ubiquitinate the BRCA2 protein, thereby achieving a result of degrading the BRCA2 protein in prostate cancer.

In the disclosure, the peptide is delivered to prostate cancer cells by using nano-selenium, and it is found that cell proliferation can be inhibited. A half maximal inhibitory concentration (IC₅₀) of the peptide drug for prostate cancer cells C4-2 is 181 nM, and an IC₅₀ of the peptide drug for prostate cancer cells PC-3 is 227 nM. When used in combination with a PARP inhibitor, a sensitivity of the prostate cancer cells and a transplanted tumor model to the PARP inhibitor can be significantly enhanced.

In view of the function of inhibiting proliferation of the prostate cancer cells, and enhancing the sensitivity of the prostate cancer cells to the PARP inhibitor of the peptide targeting the BRCA2 protein, the disclosure provides an application method of the peptide targeting the BRCA2 protein in the preparing a drug for treating mCRPC.

In the disclosure, the prostate cancer includes one or more of the following prostate cancer cells: C4-2, PC-3, lymph node carcinoma of the prostate (LNCap), 22RV1 and DU145.

In view of the function of the peptide to degrade intracellular BRCA2 protein, the disclosure provides an application method of the peptide targeting the BRCA2 protein in preparing a reagent for degrading the BRCA2 protein.

The disclosure provides a drug for treating mCRPC, including the peptide and a delivery system.

The disclosure does not impose any special restrictions on a type of delivery system, and the delivery system well known in the art can be used, such as nano-gold, liposomes, nano-selenium or other nano delivery systems well known in the art. In the embodiments of the disclosure, nano-selenium is used as a delivery system as an example to illustrate the preparation method and efficacy of the drug. The disclosure does not impose any special restrictions on the preparation method of the nano-selenium-peptide drug, and a method of nano-selenium coupling protein well known in the art can be used. The drug has been experimentally confirmed to be non-toxic at the cellular level and animal level, and has a high safety.

The following is a detailed description of the peptide targeting the BRCA2 protein and an application method thereof, and a drug for preventing and treating prostate cancer provided by the disclosure in conjunction with the embodiments, but they should not be construed as limiting a scope of protection of the disclosure.

Description of experimental materials is as follows.

The experimental materials are divided into a synthesis part, including Fmoc amino acid purchased from Shanghai GL Biochemical, N,N-diisopropylethylamine (DIEA, C₈H₁₉N), HOBT, and HBTU purchased from Sigma-Aldrich (a subsidiary of Merck Life Sciences); and a nano-selenium preparation part, including branched polyetherimide (PEI), and tetrachloroauric acid purchased from Aladdin Reagent.

### Glossary

HR represents DNA homologous recombination repair;
Se represents a single nano-selenium delivery system;
BPD represents a finished product coupled with peptide drug and nano-selenium delivery system;
Control represents a finished product coupled with a peptide negative control and the nano-selenium delivery system;
BRCA2 represents a target protein BRCA2;
PROTAC represents an abbreviation of proteolysis-targeting chimeras;
β-actin represents actin, which is used as a loading control in western blot; and
CHX represents cycloheximide, which is a protein production inhibitor.

### Embodiment 1

A protein-assisted design is used to obtain a PROTAC peptide targeting a BRCA2 domain.

The peptide drug (synthesized in the laboratory of the inventor) is synthesized according to the amino acid sequence and by using the peptide solid phase synthesis method. The synthesis method is a general Fmoc peptide synthesis. Fmoc-protected amino acids are purchased from GL Biochemical, and HBTU and HIBT condensation agents are purchased from Suzhou Highfine Biotechnology. The synthesis method is as follows.
1) Deprotection: Fmoc-protected column and monomer must be treated with an alkaline solvent (piperidine) to remove an amino protecting group.
2) Activation and cross-linking: a carboxyl group of a next amino acid is activated by an activator. The activated monomer reacts with a free amino group to cross-link and form a peptide bond. In this step, a large amount of super-concentrated reagents is used to drive the reaction to complete. Cycle: these two steps are repeated until the synthesis is complete.
3) Elution and deprotection: the peptide is eluted from the column, and protecting groups of the peptide are eluted and deprotected by a deprotecting agent (trifluoroacetic acid, abbreviated as TFA) to obtain a crude product.

The amino acid sequence of the PROTAC peptide is LLDEEDDSEEGGSGGTSSLLFSLWE (SEQ ID NO: 1).

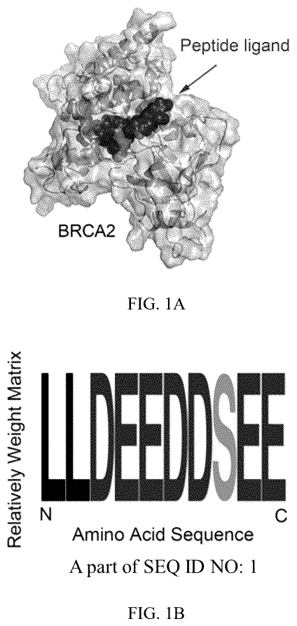

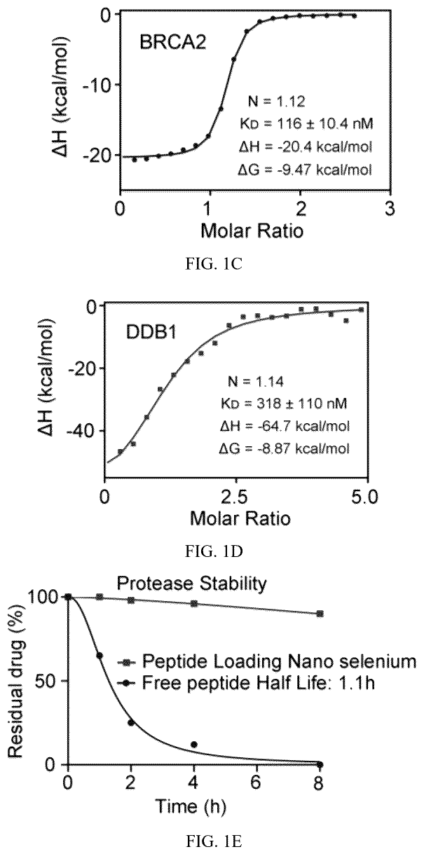

FIG. 1A is a complex structure of the peptide targeting the BRCA2 protein and the BRCA2 protein. FIG. 1B is a result of protein-assisted design of a BPD peptide. It can be seen from FIG. 1C and FIG. 1D that a dissociation constant of the BPD peptide with the BRCA2 protein is in a range of 105.6 nM to 126.4 nM, and a dissociation constant of the BPD peptide with a DDB1 protein is in a range of 208 nM to 428 nM. It can be seen from FIG. 1E that the BPD has stronger protein stability relative to common peptides.

### Embodiment 2

Preparation of a Se-BRCA2 PROTAC drug.

A coupling method is as follows. 2 milligrams (mg) of peptide is dissolved in 1 milliliter (mL) of pure water to obtain a peptide solution. 0.2 mL sodium selenite with a concentration of 50 millimoles per liter (mM), 0.6 mL chitosan with a concentration of 5% and 1.6 mL ascorbic acid with a concentration of 50 mM are added into the peptide solution to obtain a mixture. The mixture is heated to 50 Celsius degrees (° C.) to react for 20 minutes (min) to obtain a reacted mixture, and the reacted mixture is cooled to room temperature to obtain the BPD peptide coupled with nano-selenite.

### Embodiment 3

### Cell-Level Experiments on BPD

1. A cell culture method is as follows. Culture conditions of C4-2, PC-3, LNCap and 22RV1 cell lines are 1640 medium, 10% fetal bovine serum, 5% carbon dioxide (CO₂) saturated humidity, 37° C. adherent culture, and a maximum cell culture density of 90% confluence. Culture conditions of a DU145 cell line are Dulbecco's modified Eagle medium (DMEM), and the other conditions are the same as above.
2. Experiment on the ability of drugs to inhibit cancer cell proliferation.

A 3-(4,5-dimethylthiazol-2-yl)-2,5-diphenyltetrazolium bromide (MTT) detection method is used to analyze the ability of drugs to inhibit cancer cell proliferation.

MTT is a rapid and highly sensitive test kit widely used for cell proliferation and cytotoxicity. In a presence of an electron coupling reagent, MTT can be reduced by some dehydrogenases in mitochondria to generate blue-purple crystalline formazan. The more and faster the cells proliferate, the darker the color. For the same cells, a depth of the color is linearly related to the number of cells.

In the experiment of detecting cell activity, the C4-2 and PC-3 cells are inoculated into a 96-well plate treated with tissue culture (TC) at a density of 3×10⁴ cells/mL, and each well is added with 100 microliters (μL) of cell sap. The cells are subjected to adherent culture for 24 hours (h), and then treated with drugs in different concentrations (10 nM to 2000 nM). A negative control is set as the finished product coupled with a peptide negative control and the nano-selenium delivery system, and blank groups, that is, an untreated group and a background group, are set, which are only added with cell culture medium and not added with cells. After 48 h of treatment, the medium is replaced in each well, 200 μL of culture medium containing MTT is added, and the cells are incubated in a 37° C. incubator for 4 h. After the color development is completed, an absorbance of each well at 450 nanometers (nm) and 690 nm is measured by using a spectrophotometer for detection of a microplate reader. After the measurement, the absorbance of each well is calibrated according to a formula I. Finally, a cell viability is calculated according to a formula II. The formula I and the formula II are expressed as follows:

$$A = {OD}_{450} - {OD}_{690} ; \qquad Formula I$$

$$Cell viability ( \% ) = ( A ( drug added ) - A ( background ) / A ( control ) - A ( blank ) \times 100. \qquad Formula II$$

After drug treatment, the detection and calculation show that the finished product coupled with the peptide drug and the nano-selenium delivery system has an inhibitory effect on the proliferation of the prostate cancer cells.

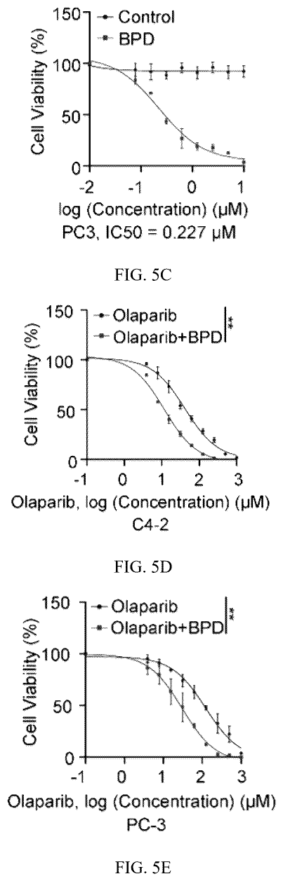

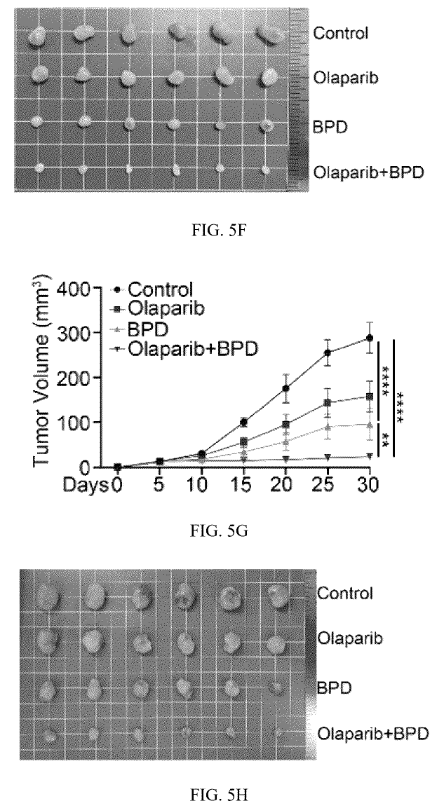

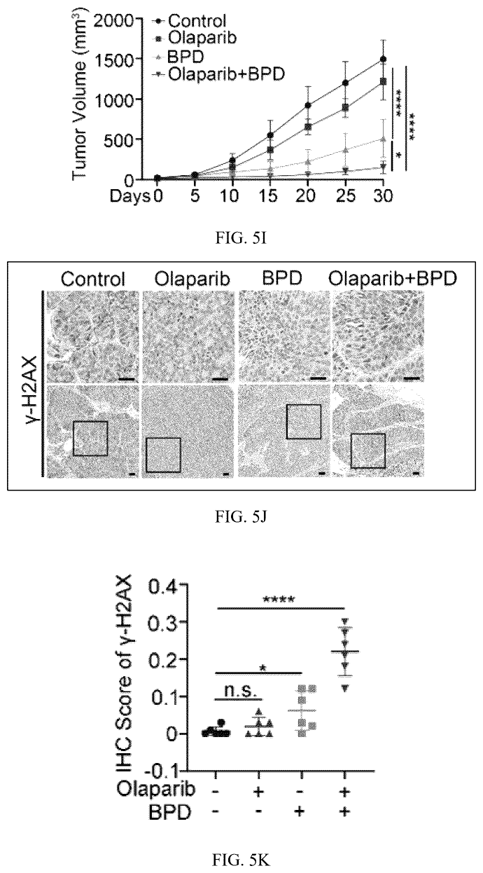

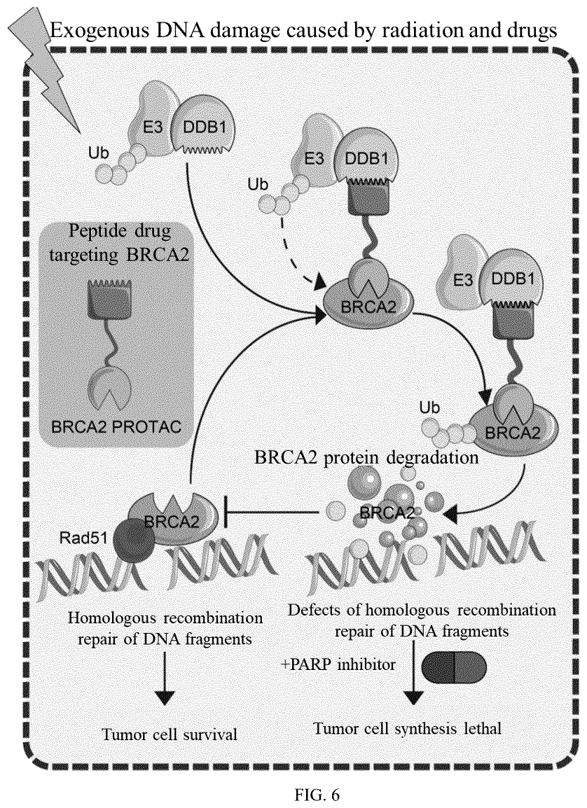

The results are shown in FIGS. 5B, 5C, 5D and 5E. The BPD shows a dose-dependent growth inhibitory effect in both C4-2 and PC-3 cells, while the peptide control has no effect on the prostate cancer cells. The IC₅₀ values of the BPD for the C4-2 and PC-3 cells are 181 nM and 227 nM, respectively. After BPD combined with a PARP inhibitor to treat cells, a growth inhibitory effect of the PARP inhibitor on cells is significantly enhanced.
3. In order to study the degradation ability of the BPD on the BRCA2 protein, immunoblotting (IB) is used to analyze the BRCA2 protein. The specific experimental process is as follows.
1) The C4-2/PC-3 cells are inoculated into a 12-well plate treated with TC at a density of 3×10⁴ cells/mL, and each well is added with 1 mL culture medium. After the cells are subjected to adherent culture for 24 h, different drugs are added into the cultured cells for treatment. After 48 h of treatment, each well is added with a radio immunoprecipitation assay (RIPA) lysis solution containing 50 μL of protease inhibitors, and the lysate is collected.
2) A total protein content in each group of samples is quantified by using a bicinchoninic acid (BCA) quantification kit, and a protein concentration in each group of samples is made consistent by adjusting a sample volume. After the protein amount is adjusted, a loading buffer is added into each group of samples, and the samples are incubated at 100° C. for 10 min to completely denature the protein.
3) Samples from different groups are subjected to sodium dodecyl sulfate-polyacrylamide gel electrophoresis (SDS-PAGE) separation. 8% polyacrylamide resolving gel containing sodium dodecyl sulfate (SDS) and 5% polyacrylamide stacking gel are prepared. Then the prepared samples and pre-stained protein samples with a same volume are added into loading wells for electrophoresis separation experiments. The electrophoresis conditions are a voltage of 70 volts (V), and a separation time of 15 min until bromophenol blue reaches the resolving gel. Then the voltage is adjusted to 120 V, the separation time is about 60 min until the bromophenol blue reaches about 1 centimeter (cm) from an end of the resolving gel, and the electrophoresis is stopped.
4) The protein samples are transferred to a membrane. All western blot experiments in the disclosure use polyvinylidene fluoride (PVDF) membranes, and three layers of filter papers, a PVDF membrane, gel, and three layers of filter papers are placed on a membrane transfer instrument in that order. A membrane transfer current is set as 300 milliamperes (mA), and a membrane transfer time is set as 2 h.
5) Blocking: the transferred PVDF membrane is immersed in a blocking solution containing 5% BSA and incubated at room temperature for 1 h to remove the influence of nonspecific adsorption.
6) Primary antibody incubation: different antibody dilutions are prepared as required, and then incubated at 4° C. overnight to achieve a purpose of antibody recognition of specific antigens.
7) Secondary antibody incubation: species-specific horseradish peroxidase (HRP)-labeled secondary antibodies (anti-mouse or anti-rabbit) are prepared according to the species of the primary antibody, diluted according to a ratio of 1:2000, and incubated at room temperature for 1 h.
8) Color development: a substrate solution is prepared to infiltrate the PVDF membrane that has been fully incubated with the secondary antibody, and a chemiluminescence analyzer is used for color development analysis.

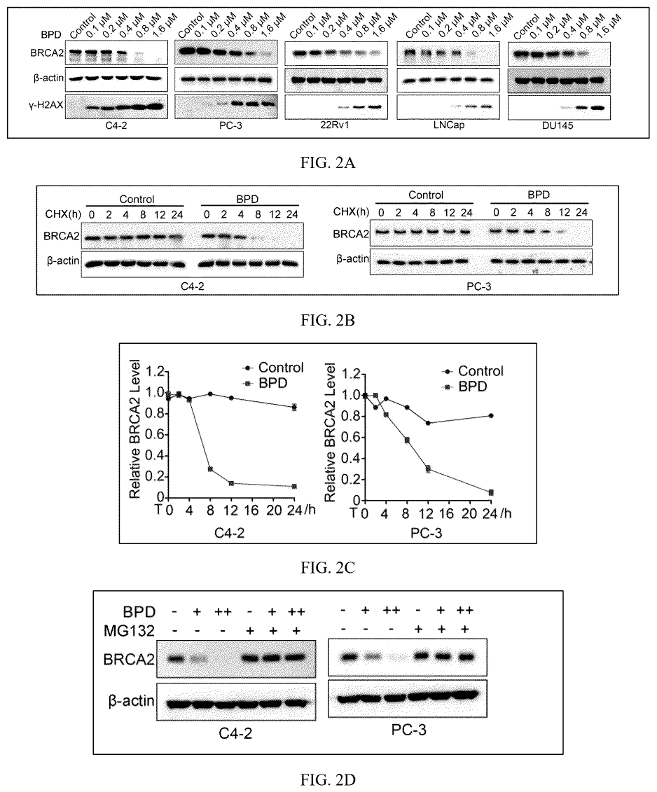

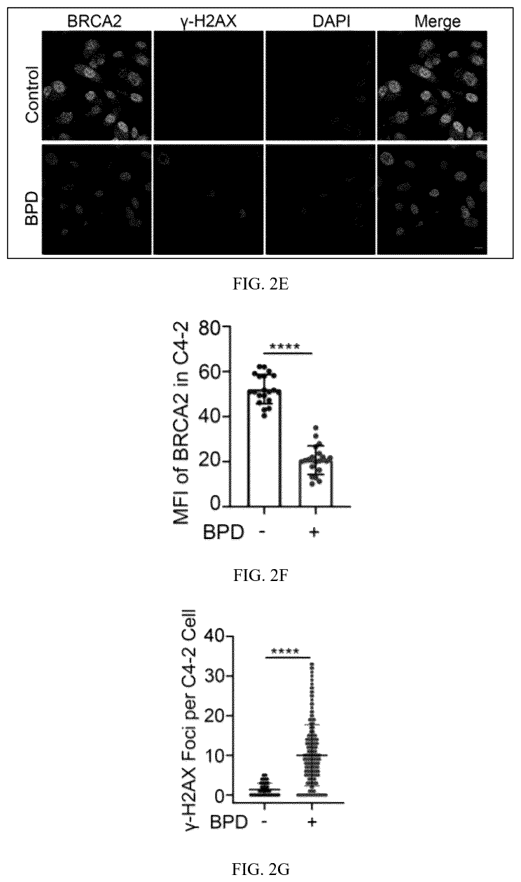

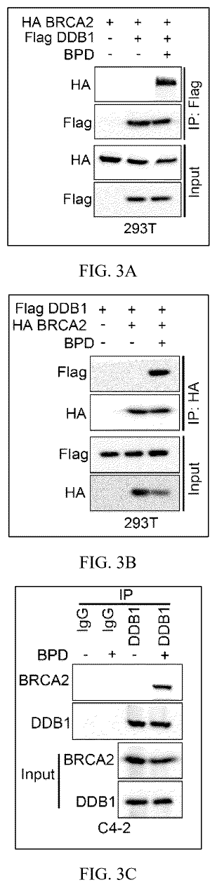

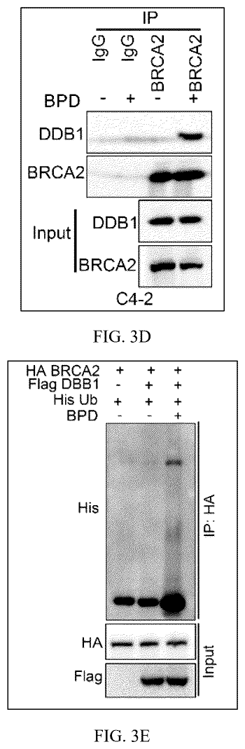

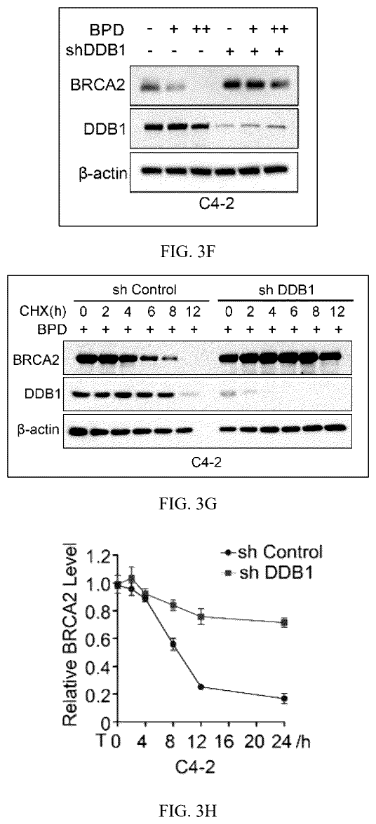

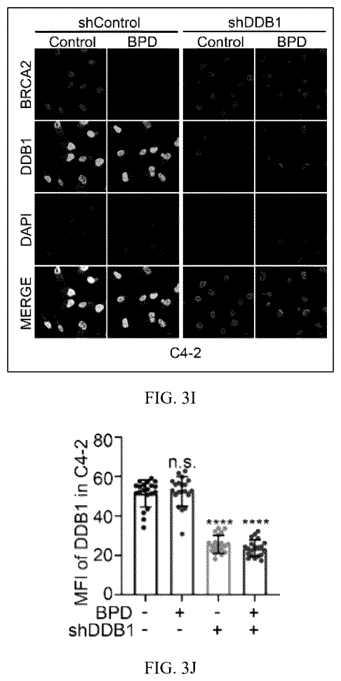

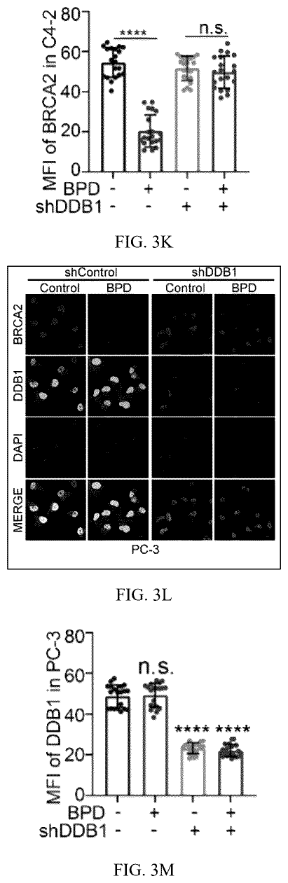

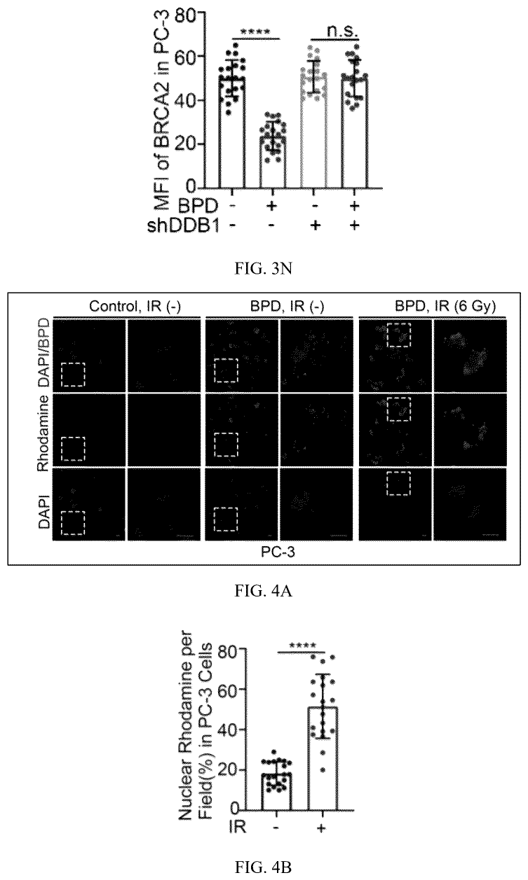

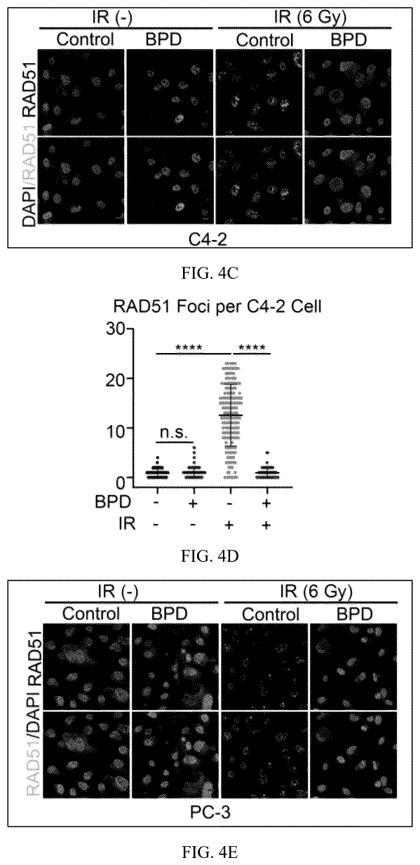

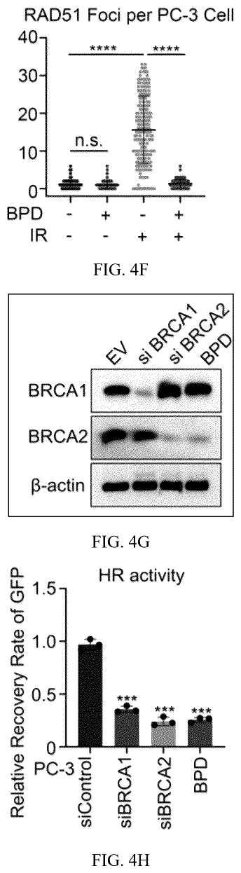

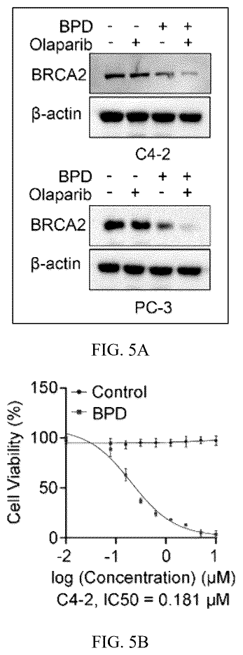

The results are shown in FIGS. 2A to 2D. The BPD induces the degradation of the BRCA2 protein in C4-2, PC-3, 22Rv1, LNCap and DU145 cells in a dose-dependent and time-dependent manner, while the peptide controls have no effect on cancer cells. As shown in FIGS. 3F to 3H, the degradation ability of the BPD on the BRCA2 protein is inhibited after knocking-down an expression level of DDB1 gene. As shown in FIG. 4G, si BRCA1 can inhibit an expression level of a BRCA1 protein, while si BRCA2 and BPD can effectively reduce an expression level of the BRCA2 protein. As shown in FIG. 5A, the PARP inhibitor enhances the degradation effect of the BPD on the BRCA2 protein in the prostate cancer cells.

### Embodiment 4

In order to study the mechanism of action of the BPD, the binding ability of the BRCA2 protein to the DDB1 protein in C4-2, PC-3 and 293T cell lines is analyzed after BPD treatment.

An immunoprecipitation (IP) experiment method is as follows.
(1) The C4-2 and PC-3 cells treated with the control peptide drugs or the BPD or cells transfected with the BRCA2 protein, the DDB1 protein and Ub plasmids are harvested, and an appropriate amount of IP lysis solution (containing proteasome inhibitors) is added to obtain a first mixture. The first mixture is lysed on ice or at 4° C. for 30 min, and then centrifuged at 12,000 gravitational accelerations (g) to collect a supernatant.
(2) A small amount of the supernatant is taken out for western blot analysis. 1 microgram (μg) of antibody and 10 μL to 50 μL of protein A/G beads are added to the remaining supernatant and incubated slowly overnight to obtain a second mixture.
(3) After the immunoprecipitation reaction, the second mixture is centrifuged at 3000 g for 5 min at 4° C. to centrifuge the protein A/G beads to a bottom of the tube. The supernatant is carefully aspirated, and the protein A/G beads are washed 3-4 times with 1 mL of the IP lysis solution to obtain washed protein A/G beads. Finally, a loading buffer is added to the washed protein A/G beads, and they are incubated at 100° C. for 10 min.
(4) Western blot analysis is performed by using the same experimental method as above.

The results are shown in FIGS. 3A to 3E. In the presence of the BPD, the BRCA2 protein and the DDB1 protein are tightly bound. The enhancement of ubiquitination is only in the presence of the BPD, indicating that the enhancement of ubiquitination is through enhancing the binding of E3 ligase DDB1 to the BRCA2 protein.

### Embodiment 5

In order to study a degradation effect of the BPD drug on BRCA2 and the effect on a HR function of cell DNA damage, the BRCA2 protein, γ-H2AX and a RAD51 protein are analyzed by using a cellular immunofluorescence assay. The specific experimental process is as follows.
(1) Coverslip cell culture: C4-2 and PC-3 cells are inoculated into a culture dish with a pre-treated coverslip at a density of 3×10⁴ cells/mL. After treatment with peptide control or the drug, the coverslip is removed and washed twice with phosphate-buffered saline (PBS).
(2) Fixation: 4% paraformaldehyde is used to fix cells on the coverslip to obtain fixed cells. The fixed cells are washed with PBS three times to obtain washed cells.
(3) Permeabilization: 0.2% non-ionic surfactant or emulsifier (such as Triton™-X100) is used to treat the washed cells for 15 min, and washed with PBS three times to obtain permeabilized cells.
(4) Blocking: 0.5% BSA is used to block the permeabilized cells for 30 min to obtain blocked cells.
(5) Primary antibody binding: the blocked cells are incubated at room temperature for 1 h or at 4° C. overnight, and then rinsed with phosphate-buffered saline polysorbate 20 (TWEEN®-20) (PBST) three times.
(6) Secondary antibody binding: the HRP-labeled secondary antibody labeled with fluorescein isothiocyanate (FITC) or sulfo-cyanine3 (Cy3) fluorescent markers is incubated at room temperature in the dark for 1 h, and rinsed with PBST three times.
(7) Mounting and testing: a mounting medium is added into the coverslip, the sample is mounted, and a fluorescence microscope is used for testing.

After treatment with the BPD, the DNA HR repair function mediated by the BRCA2 protein in the prostate cancer cells is inhibited and the DNA damage level in the cells increases.

As shown in FIG. 2E, after treatment with BPD, the expression level of fluorescently labeled BRCA2 protein in the prostate cancer cells decreases significantly, indicating that BPD can effectively degrade the BRCA2 protein. At the same time, as shown in FIGS. 2F and 2G, BPD triggers an increase in the level of γ-H2AX focus formation in cells. As shown in FIGS. 3I to 3N, after knocking-down DDB1 in the C4-2 and PC-3 cell lines, the BPD no longer triggers a decrease in the expression level of the fluorescently labeled BRCA2 protein, indicating that the degradation of the BRCA2 protein by the BPD is dependent on the DDB1 protein. As shown in FIGS. 4A to 4H, exogenous DNA damage caused by radiotherapy promotes the entry of the BPD into the cell nucleus, and the BPD inhibits the recruitment of a BRCA2 downstream protein RAD51 to form foci under DNA damage conditions.

### Embodiment 6

### Verification of the Inhibitory Effect of BPD on Tumor Growth at the Animal Level

### 1. Mouse Prostate Cancer Xenograft Model Method

In order to evaluate a therapeutic effect of the BPD in vivo, a subcutaneous transplanted prostate cancer mouse model and a patient-derived xenografted subcutaneous tumor mouse model are established. The mice are randomly divided into 4 groups and treated with the peptide control, the BPD (5 milligrams per kilogram, abbreviated as mg/kg), and Olaparib (PARP inhibitor, 50 mg/kg) with a same dose every 3 days for 3 consecutive weeks. The tumor volume is continuously observed and recorded. When the tumor volume grows to 50 cubic millimeters (mm³) to 100 mm³, the drug is injected into the mice. The tumor volume calculation is completed according to a formula III. All drugs are injected every other day, an injection method is intraperitoneal injection, an injection volume is 100 μL, and changes in tumor size are recorded at the same time. After 21 days of drug treatment, the mice are dissected, and the tumor sites and other major organs are removed for hematoxylin-eosin (HE) staining and γ-H2AX immunohistochemical staining experiments. The formula III is expressed as follows:

$$Tumor volume = ( V ) = length \times {width}^{2} / 2. \qquad Formula III$$

### 2. HE Staining Method

### 1) Tissue Fixation

The removed fresh tissues are placed in a pre-prepared 10% Formalin® (a saturated solution of formaldehyde gas in water) fixative to denature and coagulate the proteins in the tissues and cells to prevent autolysis of the cells after death or decomposition by bacteria, thereby maintaining the original morphological structure of the cells.

### 2) Tissue Dehydration

The fixed tissues each are trimmed into 25 pixels (px)×25 px×5 px, and the fixative is removed from the tissue by washing with pure water. Then the water in the tissue is gradually replaced with alcohol from low to high concentrations. The tissue block is then placed in a transparent agent, xylene, that is soluble in both alcohol and paraffin, and the alcohol in the tissue block is replaced with xylene before paraffin embedding.

### 3) Tissue Embedding

The transparent tissue block is placed in a melted paraffin, and the paraffin with the transparent tissue block is put in a paraffin melting box to keep warm. After the paraffin is completely immersed into the tissue block, let it cool and solidify into a block.

### 4) Tissue Sectioning

The embedded paraffin block is fixed on a microtome and cut into thin slides, which are generally 5 microns (μm) to 8 μm thick.

### 5) Slide Staining

Before tissue staining, the paraffin in the slides needs to be removed with xylene, and then the alcohol is removed from the slides by immersing in pure water through high-concentration to low-concentration alcohol. Then staining is started, the slides are placed in a hematoxylin aqueous solution for staining for 10 min. Then the slides are placed in acid water and ammonia water for a few seconds each for differentiation. After rinsing with pure water, the slides are dehydrated in 70% and 90% alcohol for 10 min each, and then the slides are stained in alcohol eosin staining solution for 2 min.

### 6) Dehydrate the Slides Again

The stained slides are dehydrated again according to the above tissue dehydration method.

### 7) Mounting the Slides.

The transparent slides are dripped with gum and covered with a coverslip. Then, the slides are observed under a microscope and photographed.

### 3. γ-H2AX Immunohistochemical Staining Experiment

### 1) Immunohistochemistry PBS Reagent Formula and Preparation Method

The following formula is used as an example to prepare 1000 mL of a potassium-free PBS buffer with a concentration of 0.01 moles per liter (M), and pH=7.4 for immunohistochemical staining:

| | | |
|---|---|---|
| Di-Sodium hydrogen phosphate dihydrate | 1.2 | g |
| (Na₂HPO₄•2 H₂O) | | |
| Sodium dihydrogen phosphate dihydrate | 0.5 | g |
| (NaH₂PO₄•2 H₂O) | | |
| Sodium chloride (NaCl) | 7.6 | g |
| Double-distilled water (ddH₂O) | 1000 | mL |

### 2) Immunohistochemistry Antigen Retrieval Solution Formula and Preparation Method

The following formula is used as an example to prepare 1000 mL of a citrate buffer with a concentration of 0.01 M, and pH=6.0 as an immunohistochemical antigen retrieval solution:

| | | |
|---|---|---|
| Trisodium citrate dihydrate | 3 | g |
| (C₆H₅Na₃O₇•2H₂O) | | |
| Citric acid monohydrate | 0.4 | g |
| (C₆H₈O₇•H₂O) | | |
| ddH₂O | 1000 | mL |

### 3) Graded Alcohol Preparation (100 mL)

| Graded alcohol | Anhydrous alcohol ratio |
|---|---|
| Anhydrous alcohol | 100 mL |
| 95% alcohol | 95 mL + ddH₂O (5 mL) |
| 90% alcohol | 90 mL + ddH₂O (10 mL) |
| 85% alcohol | 85 mL + ddH₂O (15 mL) |
| 80% alcohol | 80 mL + ddH₂O (20 mL) |
| 75% alcohol | 75 mL + ddH₂O (25 mL) |
| 70% alcohol | 70 mL + ddH₂O (30 mL) |
| 50% alcohol | 50 mL + ddH₂O (50 mL) |

### 4) Tissue Fixation, Sectioning and Baking

Tissue fixation and sectioning are performed in the same manner as described above for HE staining, and then the tissue slides are placed in an oven and baked at 60° C. for 2 h.

### 5) Dewaxing

The slides are removed from the oven and immediately placed in xylene for dewaxing:

| | |
|---|---|
| Xylene I | 8 min |
| Xylene II | 8 min |
| Xylene III | 8 min |

6) Hydration with Graded Alcohol

The slides are removed from xylene III and immediately placed in graded alcohol for hydration:

| | |
|---|---|
| Anhydrous alcohol I | 8 min |
| Anhydrous alcohol II | 8 min |
| 95% alcohol I | 3 min |
| 95% alcohol II | 3 min |
| 85% alcohol | 3 min |
| 80% alcohol | 3 min |
| 75% alcohol | 3 min |
| 70% alcohol | 3 min |
| 50% alcohol | 3 min |

### 7) PBS Washing

The slides are removed from the 50% alcohol and immediately washed in PBS three times for 3 min each time, to thereby remove the alcohol.

### 8) Antigen Retrieval

The slides are removed from PBS and placed in a histochemical slide box filled with 0.01 M, pH=6.0 citrate buffer, then the histochemical slide box is placed in a digitally controlled hot antigen repair instrument for antigen retrieval at 121° C. for 5 min. After antigen retrieval is completed, the pressure is naturally reduced, then the histochemical slide box is taken out and cooled naturally to room temperature.

### 9) PBS Washing

The slides are removed from the citrate buffer and immediately washed in PBS three times for 3 min each time, to thereby remove the citrate buffer.

### 10) PBS Washing

The slides are washed in PBS three times for 3 min each time, to thereby remove the endogenous peroxidase.

### 11) Endogenous Peroxidase Blocking

The slides are removed from PBS, the PBS around the tissue slides is wiped with a filter paper, and then a hydrophobic barrier pen (e.g., PAP pen) is used to outline an outside of each tissue slide. A pipette is used to absorb 1 mL of nonspecific blocking agent to cover the entire tissue on the tissue slide, and let it stand at room temperature for 30 min.

### 12) PBS Washing

The slides are washed in PBS three times for 3 min each time, to thereby remove the nonspecific blocking agent.

### 13) Primary Antibody Incubation

According to an antibody specification, the primary antibody is diluted to an appropriate concentration with a primary antibody diluent. The slides are removed from the PBS, the PBS around the tissue slides is wiped with the filter paper, and then the hydrophobic barrier pen is used to outline the outside of each tissue slide. The pipette is used to absorb 1 mL of the prepared primary antibody solution to cover the entire tissue on the tissue slide, and the primary antibody diluent is set as a negative control (i.e., no primary antibody is added to the primary antibody diluent). The slides with the primary antibody are placed in an immunohistochemical wet box, and the immunohistochemical wet box with the slides is placed in a 4° C. medical refrigerator to stand overnight.

### 14) Rewarming

The immunohistochemical wet box is taken out from the 4° C. medical refrigerator and placed at room temperature for 1 h.

### 15) PBS Washing

The slides are washed in PBS three times for 3 min each time, to thereby remove the primary antibody solution.

### 16) Secondary Antibody Incubation

The slides are removed from PBS, the PBS around the tissue slides is wiped with the filter paper, and then the hydrophobic barrier pen is used to outline the outside of each tissue slide. The pipette is used to absorb a secondary antibody solution to cover the entire tissue on the tissue slide, and the slides with the secondary antibody are placed in an immunohistochemical wet box, and the immunohistochemical wet box with the slides is placed at room temperature for 1 h.

### 17) PBS Washing

The slides are washed in PBS three times for 3 min each time, to thereby remove the secondary antibody solution.

18) 3,3′-diaminobenzidine (DAB) Color Development

The slides are removed from the PBS, the PBS around the tissue slides is wiped with the filter paper, and then the hydrophobic barrier pen is used to outline the outside of each tissue slide. The pipette is used to absorb 1 mL of DAB dye solution (DAB: substrate buffer=26 μL: 1000 μL) to cover the entire slide. Different primary antibodies have different color development times. A termination time is determined under a microscope, and the slides are placed into deionized water to terminate the staining.

### 19) PBS Washing

The slides are washed in PBS three times for 3 min each time, to thereby remove the DAB dye solution.

### 20) Re-Staining

The slides are removed from the PBS, the PBS around the tissue slides is wiped with the filter paper, and then the hydrophobic barrier pen is used to outline the outside of each slide. The pipette is used to absorb 1 mL of hematoxylin stain to cover the entire slide. The slides with the hematoxylin stain are left at room temperature for 5 min, and placed into the deionized water to stop staining.

### 21) PBS Washing

The prostate cancer clear cell carcinoma tissue chips (i.e., the slides) are washed in PBS three times for 3 min each time, to thereby remove the hematoxylin staining.

### 22) Hydrochloric Acid Differentiation

The slides are removed from the PBS, the PBS around the tissue slides is wiped with the filter paper, and then the hydrophobic barrier pen is used to outline the outside of each slide. The pipette is used to absorb 1 mL of hydrochloric acid solution to cover the entire slide. The slides with the hydrochloric acid solution are left at room temperature for 5 seconds(s), and then placed into the deionized water to stop staining.

### 23) Ammonia Bluing

After the slides are differentiated, they are immediately placed in 1% ammonia solution. The ammonia solution with the slides is left at room temperature for 10 s, and then placed in the deionized water to terminate the reaction.

24) Dehydration with Graded Alcohol and Clearing with Xylene

The prostate cancer clear cell carcinoma tissue chips (i.e., the slides) are placed in graded alcohol and xylene solutions for dehydration:

| | |
|---|---|
| 50% alcohol | 3 min |
| 70% alcohol | 3 min |
| 75% alcohol | 3 min |
| 80% alcohol | 3 min |
| 85% alcohol | 3 min |
| 95% alcohol I | 3 min |
| 95% alcohol II | 3 min |
| Anhydrous alcohol I | 8 min |
| Anhydrous alcohol II | 5 min |
| Xylene I | 8 min |
| Xylene II | 8 min |
| Xylene III | 8 min |

### 25) Mounting

The slides are taken out from xylene III, and after the xylene evaporates, neutral gum is added to an area where the tissues are located, and the tissues are covered with an 8 cm×8 cm coverslip and placed in a fume hood to air dry, and then observed under a microscope and photographed.

As shown in FIG. 5F to 5I, nano-selenium particles have no therapeutic effect on tumor growth. The BPD shows high efficacy in both PC-3 xenograft model and patient-derived xenograft subcutaneous tumor model. Olaparib (the PARP inhibitor) also shows similar results of inhibiting tumor growth as low-concentration BPD in both models. The combined use of BPD and Olaparib shows a more efficient effect of inhibiting tumor growth in both models. Immunohistochemistry (IHC) analysis of γ-H2AX in each group in the patient-derived xenograft subcutaneous tumor model confirms that BPD can effectively induce DNA damage in tumor cells (FIGS. 5J and 5K).

Although the embodiments of the disclosure have been described, those skilled in the art may make other changes and modifications to these embodiments once they have learned a basic creative concept. Therefore, the appended claims are intended to be interpreted as including the embodiments and all changes and modifications that fall within a scope of the disclosure.

Apparently, those skilled in the art can make various changes and modifications to the disclosure without departing from a spirit and a scope of the disclosure. Thus, if these modifications and variations of the disclosure fall within a scope of the claims of the disclosure and their equivalents, the disclosure is also intended to include these modifications and variations.

What is claimed is:

1. A peptide targeting a breast cancer gene 2 (BRCA2) protein, wherein the amino acid sequence of the peptide is as shown in SEQ ID NO: 1.

2. A drug for treating prostate cancer, comprising the peptide as claimed in claim 1 and a nano-selenium delivery system.

3. The drug for treating the prostate cancer as claimed in claim 2, further comprising a poly(adenosine diphosphate-ribose) polymerase (PARP) inhibitor.

4. The drug for treating the prostate cancer as claimed in claim 3, wherein the PARP inhibitor comprises Olaparib.

5. An application of the drug for treating the prostate cancer as claimed in claim 2, comprising:
preparing a reagent for degrading the BRCA2 protein by using the drug for treating the prostate cancer.

6. An application of the drug for treating the prostate cancer as claimed in claim 2, comprising:
preparing the drug for treating the prostate cancer.

7. The application as claimed in claim 6, wherein the prostate cancer is metastatic castration-resistant prostate cancer (mCRPC).


SEQUENCE LISTING (1 sequence)

SEQ ID NO: 1 - protein, 25 aa, synthetic construct
LLDEEDDSEEGGSGGTSSLLFSLWE